(19) United States
(12) Patent Application Publication
(10) Pub. No.: US 2016/0166771 A1
(43) Pub. Date: Jun. 16, 2016

(54) Aspirating Hypodermic Syringe and Syringe Adapter

(71) Applicant: Jane Taylor, New York, NY (US)

(72) Inventor: Jane Taylor, New York, NY (US)

(21) Appl. No.: 14/570,215

(22) Filed: Dec. 15, 2014

(51) Int. Cl. A61M 5/31

(52) U.S. Cl. CPC A61M 5/3148; A61M 5/3137

(57) ABSTRACT

Embodiments of the invention relate to syringes that are adapted to make it easier for a user to aspirate fluids into the syringes. According to one embodiment, a syringe includes a plunger with a thumb rest at one end. One or more spacers extend from the thumb rest, and shelf that incorporates a distal thumb pushback surface may also be attached to the spacers. The relative configuration of the thumb rest, the spacers, and the shelf are such that a user may easily insert a thumb between the thumb rest and the shelf and may aspirate by raising the thumb above the thumb rest and pushing with the dorsal thumb against the pushback surface.

Alternative embodiments of the invention may include an adapter configured to be attached to a plunger that lacks a dorsal thumb pushback surface, thereby allowing a user to conveniently use such a syringe for aspiration.

### BACKGROUND

The basic functions of a syringe include aspirating and injecting fluids. To aspirate, a user may withdraw a plunger from the barrel of the syringe, creating a vacuum that fluids may be drawn into. To inject, a user may push the plunger back into the barrel, expelling a fluid.

One use of aspiration is to determine whether the tip of a hypodermic syringe's needle has lodged in a blood vessel, e.g., immediately before injecting the contents of the syringe into the patient. For example, in medicine and dentistry, when injecting local anesthetic into a patient, one must take care not to inject the anesthetic into a blood vessel. To do this, the user may insert the syringe, loaded with anesthetic, into the tissue that is to be numbed. The injector may then pull the plunger backwards slightly to aspirate fluid into the syringe. If blood flows (or “refluxes”) into the syringe, the needle is in a blood vessel, and the injection should not proceed until the user repositions the needle.

Aspiration can be awkward with many common syringes, though. For example, positioning the user's thumb for aspiration may require such dexterity that the step seems a nuisance. This inconvenience may tempt providers occasionally to skip refluxing before injecting fluids, e.g., anesthetics, increasing the risk of serious complications.

### BRIEF SUMMARY OF THE INVENTION

Embodiments of the invention relate to syringes that are adapted to make it more convenient and less awkward for a physician to withdraw the plunger in a controlled manner, using only the hand that is also holding the syringe. (This discussion may refer primarily to hypodermic syringes, but the invention is of general applicability.) In embodiments of the invention, this adaptation may take the form of a thumb grip that is incorporated into the plunger and placed so that a user may withdraw the plunger by raising the thumb to exert force against the thumb grip. Embodiments of the invention also include adapters that may attach to plungers to conventional hypodermic syringes to provide thumb grips with corresponding functions.

According to an embodiment of the invention, an aspirating hypodermic syringe comprises a barrel and a plunger. The plunger comprises a shaft that has a proximal end and a distal end. A rubber head is affixed to the distal end of the plunger and configured to form a liquid-tight seal around the inside of the barrel when the plunger is inserted into the barrel. According to this embodiment, the plunger also comprises a thumb rest at the proximal end of the shaft, a shelf incorporating a dorsal thumb pushback surface, the shelf facing and opposed to the thumb rest, and a rigid spacer maintaining the relative positions of the thumb rest and the shelf.

In an embodiment of the invention, the thumb rest, the spacer, and the shelf together form a “C” shape. In an embodiment of the invention, the plunger shaft, the thumb rest, the spacer, and the shelf are molded together as a single unit.

According to an embodiment of the invention, an adapter for a hypodermic syringe comprises a thumb-contact member, one or more spacers, each of which is attached substantially perpendicular to the thumb-contact member, and a plunger-contact member, attached substantially perpendicular to the spacers and facing and opposed to the thumb-contact member. The plunger-contact member incorporates a cutout adapted to being snapped onto a plunger of a hypodermic syringe.

In an embodiment of the invention, the adapter is molded as a single piece. In an embodiment of the invention, the adapter is shaped like a letter “C”.

According to an embodiment of the invention, an assembly includes a hypodermic syringe. The assembly comprises a barrel, a plunger, and an adapter. The plunger comprises a shaft comprising a proximal end and distal end, a rubber head affixed to the distal end of the plunger and configured to form a liquid-tight seal around the inside of the barrel when the plunger is inserted into the barrel, and a thumb rest at the proximal end of the shaft. The adapter comprises a thumb-contact member, one or more spacers, each attached substantially perpendicular to the thumb-contact member, and a plunger-contact member attached substantially perpendicular to the spacers and facing and opposed to the thumb-contact member, the plunger-contact member incorporating a cutout adapted to being snapped onto the plunger.

### BRIEF DESCRIPTION OF THE DRAWINGS

FIG. 1 depicts an aspirating hypodermic syringe according to an embodiment of the invention.

FIGS. 2-4 depict a plunger for use as part of a hypodermic syringe according to an embodiment of the invention.

FIG. 5 depicts a portion of a plunger that may be used in connection with an embodiment of the invention.

FIGS. 6-8 depict an adapter according to an embodiment of the invention.

FIG. 9 depicts an adapter and a plunger according to an embodiment of the invention.

### DETAILED DESCRIPTION OF PREFERRED EMBODIMENTS

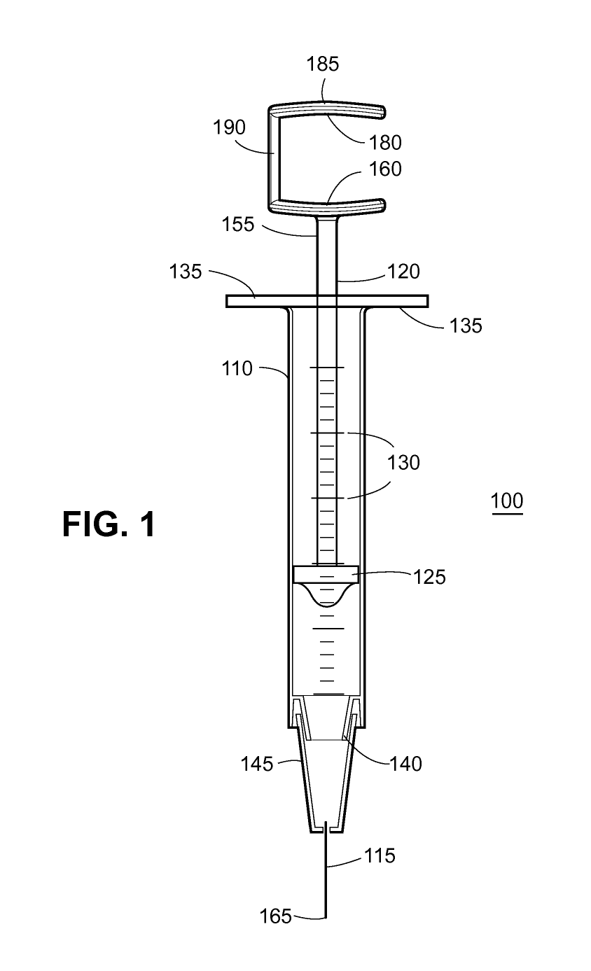

FIG. 1 depicts an aspirating hypodermic syringe 100 according to an embodiment of the invention. As depicted, the main parts of the syringe 100 are the barrel 110, the hypodermic needle 115, and the plunger 120, which incorporates the plunger cap 125.

The barrel 110 of the syringe 100 may be made, e.g., of polypropylene. The barrel 110 may be translucent or substantially transparent, allowing the user to see the amount of liquid (not pictured) in the syringe 100, and the barrel 110 may be marked, e.g., with printed and/or molded gradations 130, to allow the user to see how much liquid the syringe 100 contains. The top of the barrel 110 may incorporate two finger grips 135, which may be, e.g., flanges that may rest against the user's fingers to provide an opposing force when the user pushes the plunger 120 into the barrel 110.

The barrel 110 may incorporate a fitting 140 at the bottom, e.g., for attachment to a hypodermic needle 115. According to an embodiment of the invention, the fitting 140 may be, e.g., a male Luer lock connection such as the ISO 594 standards specify. The hypodermic needle 115, in connection with an embodiment of the invention, may incorporate a corresponding female Luer lock connection 145.

In an embodiment of the invention, a hypodermic syringe 100 comprises a plunger 120, which may, e.g., also be made of a rigid plastic such as polypropylene. As depicted, the plunger 120 comprises a rubber head 125 at the bottom of a rigid shaft 155. The end of the shaft 155 opposite the head 125 may comprise, e.g., a thumb rest 160. The thumb rest 160 is typically substantially flat, although it may also be, e.g., recessed in the center and/or otherwise convex, and/or it may be textured, e.g., to let the user comfortably and effectively press the thumb rest 160 when administering an injection and/or to keep the user's thumb from slipping.

Notwithstanding differences between embodiments of the invention and other syringes, a syringe 100 according to an embodiment of the invention may be consistent with certain uses of hypodermic syringes, e.g., as are known to those skilled in the arts. For example, according to one method of using a hypodermic syringe 100, the plunger 120 is inserted fully into the barrel 110. Holding the barrel 110 of the syringe 100 in one hand, the user immerses the end 165 of the needle 115 in a liquid, such as a drug intended for injection.

According to this method, the user then grasps the end of the plunger 120 in the other hand and pulls it out of the barrel 110. The flexible rubber head 125 forms a seal completely around the inner circumference of the barrel 110, so withdrawing the plunger 120 tends to create a vacuum inside the barrel 110, which causes fluid (not pictured) to be drawn into the barrel 110 through the hypodermic needle 115. When the user judges, e.g., from the markings 130 on the barrel 110 that the desired amount of liquid has been drawn into the syringe 100, the user stops pulling the plunger back 120 and removes the needle tip 165 from the liquid.

The user may then insert the needle 115, e.g., into the skin and subcutaneous tissues of the patient (not pictured) who is to receive the injection. Once the needle 115 has been inserted, the user may typically hold the syringe 100 by placing the barrel 110 between two fingers of one hand, with the fingers resting on opposite sides of the barrel 110, just below the finger grip 135. The user may inject the liquid by pushing with the thumb of that same hand against the surface of the thumb rest 160. The fingers provide opposing forces against the finger rests 135, preventing the barrel 110 from moving vertically in response to the force that the thumb exerts. Thus, the effect of the force that the thumb exerts is to push the plunger 120 into the barrel 110 of the syringe 100, thereby expelling the fluid from the barrel 110, e.g., through the hypodermic needle 115.

When the user's fingers are placed against the finger rests 135, and the thumb is above the thumb rest 160, regardless of whether the thumb is exerting significant force against the thumb rest 160, positioned, e.g., as for an injection as described above, the user's hand and the syringe 100 may be said to be in position for injection.

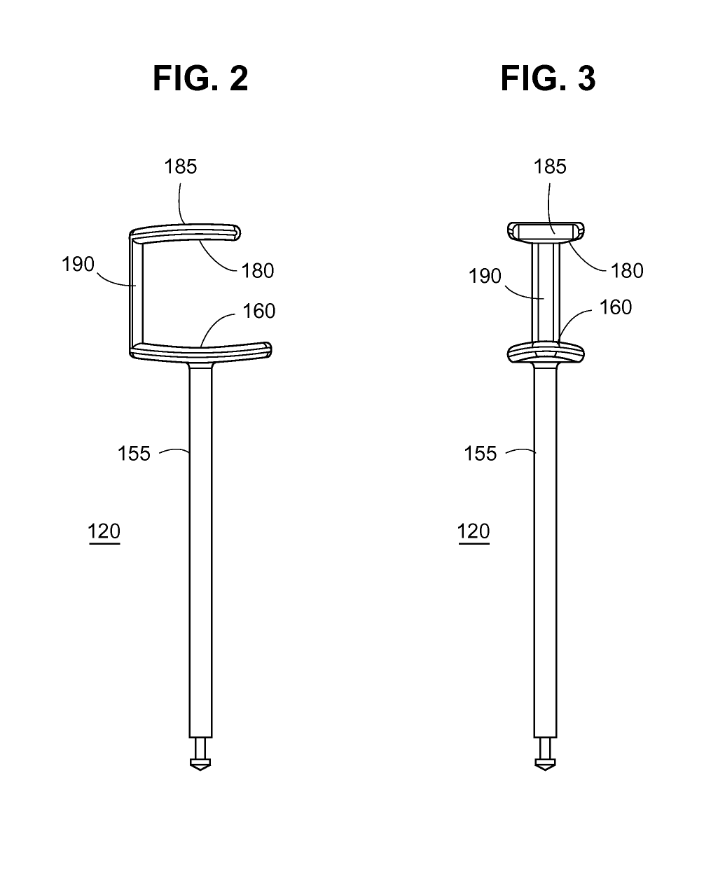

FIG. 2 depicts a plunger 120 for an aspirating syringe 100 according to an embodiment of the invention. The depicted plunger 120 includes a dorsal thumb pushback surface 180. The pushback surface 180, as FIG. 2 depicts, is slightly curved, having a slight concavity that faces the thumb rest 160. As depicted, the pushback surface 180 and the thumb rest have curves that face and mirror each other.

In an embodiment of the invention, the pushback surface 180 may be the undersurface of a shelf 185 that is separated from the thumb rest by one or more spacers 190, e.g., as FIG. 2 depicts. For example, as FIG. 2 depicts, the shelf 185 may be separated from the thumb rest 160 by a single solid spacer 190. Alternatively, one or more spacers, e.g., columnar spacers (not pictured), may maintain the separation.

An exemplary use of an aspirating syringe 100, in connection with an embodiment of the invention, may begin with the user drawing liquid into the syringe 100, inserting the needle 115 into the patient, and then holding the syringe 100 in an injection position, e.g., as described above. In such a use, the user may then attempt to reflux by raising the thumb from the thumb rest 160 and pushing against the dorsal thumb pushback surface 180 with the dorsal thumb. It will be appreciated by those skilled in the appropriate arts that the user may not need to apply very much force to the pushback surface 180 in such use: if the tip 165 of the hypodermic needle 115 is in a blood vessel, little force may be required to reflux blood from the vessel into the syringe 100, and any significant resistance that the user may feel may indicate that the tip 165 is not in a blood vessel.

According to an embodiment of the invention, the shapes, spacing, relative orientation, and composition of the elements of the plunger (viz., the shaft 155, the thumb rest 160, the spacer or spacers 190, and the pushback surface 180 and the shelf 185 it is a part of) are consistent with the above exemplary use. For example, according to an embodiment of the invention, the pushback surface 160 may be sufficiently flat so that it does not cause discomfort to the user's thumb, and sufficiently parallel to the thumb rest 160 that the user's thumb will not tend to slide out due to lateral components of the opposing force that the pushback surface 180 exerts against the thumb.

According to an embodiment of the invention, the configuration of the spacers 190 does not impede the free placement of the user's thumb between the thumb rest 160 and the pushback surface 180. For example, as FIGS. 2-4 depict, the thumb rest 160 and the pushback surface 180 are both ovals (as FIG. 4 depicts, the oval of the shelf 180 is actually cut off near one end), roughly parallel to and facing each other, each oval being curved slightly towards the other along the long axis. As depicted, a spacer 190 connects the shelf 180 to the thumb rest 160, and the spacer 190 is positioned opposite the user's thumb.

In other embodiments, the thumb rest 160 and/or pushback surface 180 may have shapes other than as FIGS. 2-4 depict, e.g., they may be circular, rectangular, or nearly so. In an embodiment of the invention, the spacer or spacers 190 may be placed only to subtend respectively portions of a hypothetical contiguous ninety-degree arc centered at or near the center of the thumb rest. So limiting the placement of the spacer or spacers 190 may in an embodiment of the invention minimize obstructions to easy placement and removal of the thumb.

The space the spacer or spacers 190 maintain the space between the top of the thumb rest 160 and the pushback surface 180. In an embodiment of the invention, this space may be wide enough to allow easy placement and removal of the user's thumb, but also narrow enough that an aspirating user need not raise the thumb excessively to contact the pushback surface 180.

For example, in one exemplary embodiment such as FIGS. 2-4 depict, the pushback surface 180 and the top of the thumb rest 160 are 20 mm apart. The shelf 185 comprising the pushback surface 180 measures 20 mm along a direction parallel to the thumb and 14 mm along a direction perpendicular to the first. The thumb rest 160 extends, e.g., 30 mm along the direction parallel to the thumb and 14 mm along the direction perpendicular to the first. The shelf, spacer, and thumb rest are each 2.5-3 mm thick.

The preceding dimensions are exemplary, not limiting, and elements of a plunger according to any embodiment of the invention may have any shapes and sizes consistent with the intended use of the syringe. Further, in an embodiment of the invention, any one or more corners of any or all elements may be rounded and/or tapered, e.g., for aesthetic reasons or the user's comfort, such as FIGS. 2-4 depict.

It will be appreciated from the above description and the accompanying figures that, in an embodiment of the invention, the thumb rest 160, the spacer 190, and the shelf 185 may form a shape that, viewed from the side, resembles a letter “C”. In describing embodiments of the invention, one or more structures may be described as “C”-shaped if they consist of one or more joined straight and/or curved segments that, together, resemble a letter “C”. (A structure is not “C”-shaped, however, if it forms a closed or nearly-closed loop such that a user's thumb may not conveniently be swung in or out of the area that the shape surrounds.)

The elements of the plunger (other than the rubber head) may be made of any material sufficiently rigid to bear and transmit the forces that may be expected in a use such as described above, possibly with some flexion. For example, one suitable material known to the art is ultra high molecular weight polyethylene (UHMWPE). Other medical-grade plastics are also suitable, including, for example, polyethylene and polypropylene, and other suitable materials will be apparent to those skilled in the art.

It will be appreciated that a plunger (again, other than the rubber head) according to an embodiment of the invention may be formed, e.g., by molding it a single piece, e.g., of plastic. Alternatively, a plunger according to an embodiment of the invention may be formed by joining one or more pieces, permanently or otherwise, e.g., by gluing, welding, and/or otherwise securing the pieces to each other.

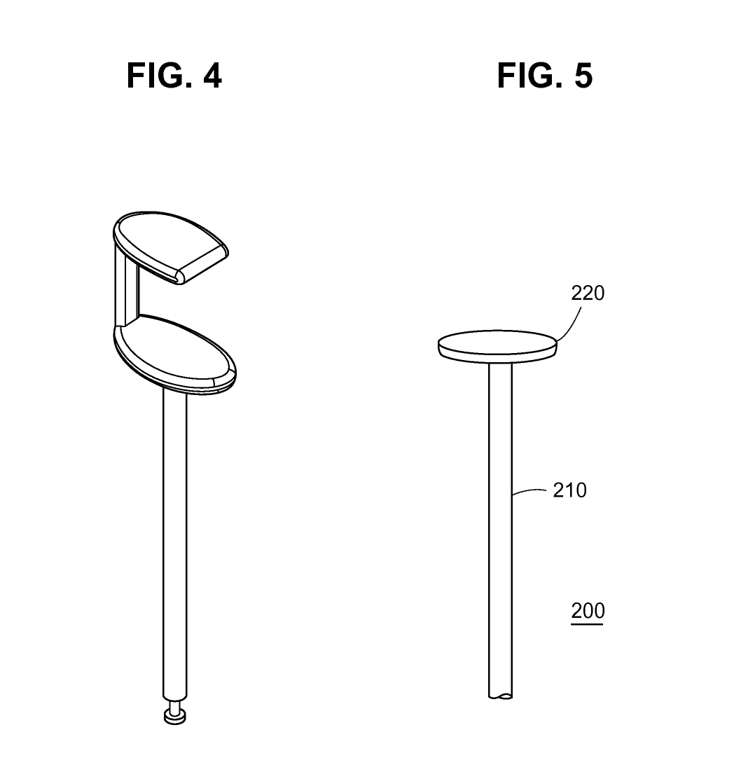

An alternative embodiment of the invention relates to providing an adapter that may be fitted to a plunger that, as manufactured, lacks a dorsal thumb pushback surface. FIG. 5 depicts the upper part of a plunger 200 such as may be used with an adapter according to an embodiment of the invention. As depicted, the plunger incorporates a cylindrical shaft 210 and a disk-shaped thumb rest 220. Although it is possible to aspirate with a syringe that incorporates a plunger 200 such as FIG. 5 depicts, doing so is relatively inconvenient: the user must hold the syringe (not pictured) steady with one hand while grasping the plunger with the other hand, which withdraws the plunger 200 from the syringe.

Thus, a plunger 200 such as FIG. 5 depicts is not adapted to make aspiration easier. Such a plunger 200 may for that reason be referred to herein as a non-aspirating plunger (even though there may be a sense in which that is not literally accurate), and a syringe that incorporates such a plunger 200 may similarly be referred to as a non-aspirating syringe.

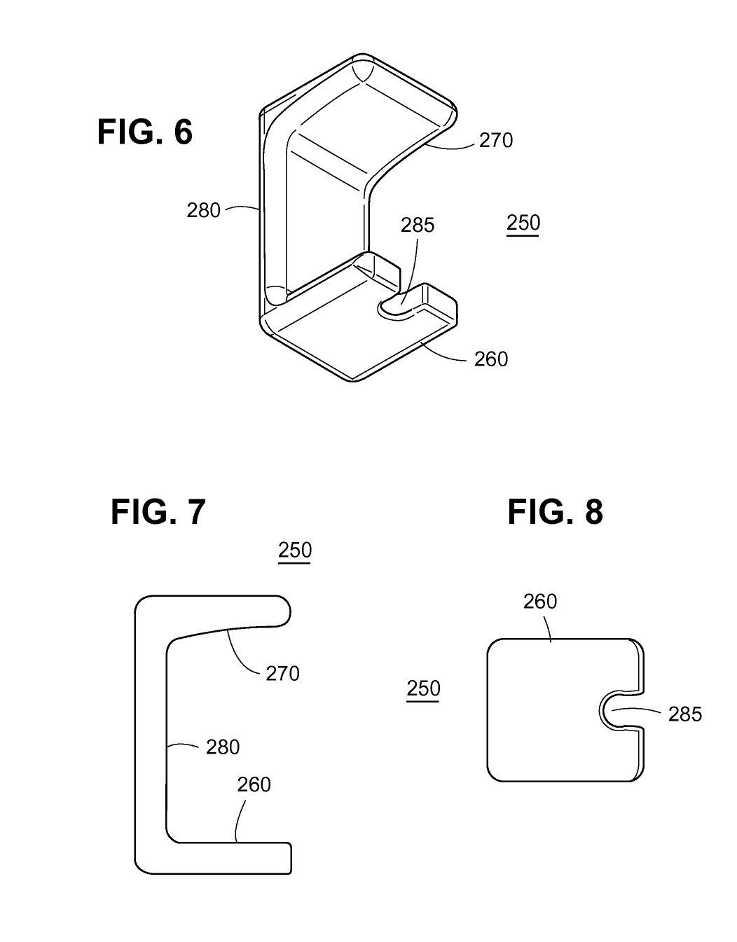

FIGS. 6-8 depict an adapter 250 for use with a non-aspirating plunger 200 according to an embodiment of the invention. The adapter 250, as depicted, is roughly C-shaped, consisting of a plunger-contact member 260 and a thumb-contact member 270, joined by a spacer 280. In the depicted embodiment of the invention, the plunger-contact member 260 and the thumb-contact member 270 are roughly parallel and opposed, and both of them extend perpendicularly from the spacer 280.

In an embodiment of the invention, the plunger-contact member 280 incorporates a cutout 285 near the center of the forward edge of the plunger-contact member 280. The cutout 285 may be shaped like a partial circle that is extended by two lines towards the edge of the plunger-contact member 280. The dimensions of the cutout may be such that the shaft 210 of a plunger 200, e.g., as FIG. 5 depicts, may be snapped into the cutout and held in place by the frictional force exerted by the inner surface of the cutout 285 pushing against the cylindrical surface of the plunger shaft 210.

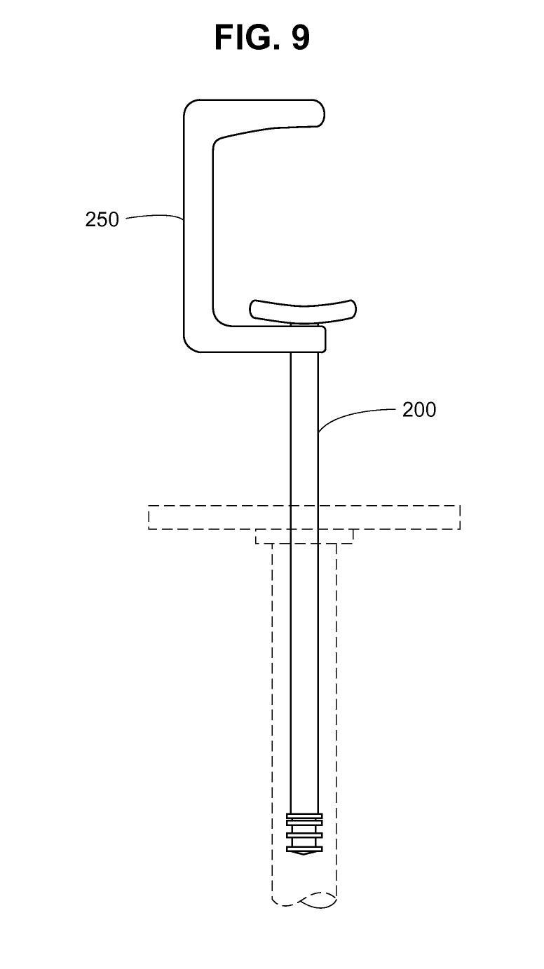

FIG. 9 depicts the relative configuration of a non-aspirating plunger 200 joined with an adapter 250 according to an embodiment of the invention. As FIG. 9 depicts, the inner surface of the plunger-contact member 260 is in contact with the underside of the thumb rest 220. The spacer 280 holds the thumb-contact member 270 above the thumb rest 220, creating space for the user's thumb. When an adapter is affixed, e.g., as FIG. 9 depicts, to a plunger in a syringe (not pictured), the user may reflux fluid into the syringe by raising the thumb under the dorsal surface of the thumb-contact member 270, pushing against that member. The user may expel fluid from the syringe by pushing down on the thumb rest 220.

The adapter 250 may be made of any material that is sufficiently rigid to transmit the forces that are applied to the adapter but also sufficiently flexible and elastic to allow the shaft 210 of the plunger 200 to be snapped into the cutout 285 and held in place by friction. Suitable materials may include, e.g., materials suitable for use in a plunger described in other embodiments of the invention. For example, one suitable material known to the art is ultra high molecular weight polyethylene (UHMWPE). Other medical-grade plastics are also suitable, including, for example, polyethylene and polypropylene, and other suitable materials are apparent to those skilled in the art.

An adapter according to an embodiment of the invention may be a single piece of molded material. Alternatively, in an embodiment of the invention, the adapter may be made of two or more pieces that are joined together.

It will be appreciated that the shape and dimensions of the cutout 285 according to embodiments of the invention may depend on the shape and dimensions of the non-aspirated plunger that the adapter is meant to be used with. And just as a plunger may have a shaft that is not cylindrical, but has some other shape, an adapter may in an embodiment of the invention have a cutout adapted to receive a shaft of such other shape.

The invention has been described in connection with specific preferred embodiments, but it should be understood that the invention as claimed is not limited to those specific embodiments. Various modifications to the described modes for carrying out the invention will be obvious to those skilled in the relevant arts, and those are intended also to be within the scope of the claims. The invention is limited solely by the claims and is not restricted to any or all described embodiments.

1. An aspirating hypodermic syringe, comprising:
a barrel; and
a plunger that comprises
a shaft comprising a proximal end and a distal end,
a rubber head affixed to the distal end of the plunger and configured to form a liquid-tight seal around the inside of the barrel when the plunger is inserted into the barrel,
a thumb rest at the proximal end of the shaft,
a shelf incorporating a dorsal thumb pushback surface, the shelf facing and opposed to the thumb rest, and
a rigid spacer maintaining the relative positions of the thumb rest and the shelf.

2. The aspirating hypodermic syringe of claim 1, wherein the thumb rest, the spacer, and the shelf form a “C” shape.

3. The aspirating hypodermic syringe of claim 2, wherein the plunger shaft, the thumb rest, the spacer, and the shelf are molded together as a single unit.

4. A plunger for an aspirating hypodermic syringe, the plunger comprising:
a shaft comprising a proximal end and a distal end;
a rubber head affixed to the distal end of the plunger and configured to form a liquid-tight seal around the inside of the barrel when the plunger is inserted into the barrel;
a thumb rest at the proximal end of the shaft;
a shelf incorporating a dorsal thumb pushback surface, the shelf facing and opposed to the thumb rest; and
a rigid spacer maintaining the relative positions of the thumb rest and the shelf.

5. The plunger of claim 4, wherein the thumb rest, the spacer, and the shelf form a “C” shape.

6. The plunger of claim 5, wherein the plunger shaft, the thumb rest, the spacer, and the shelf are molded together as a single unit.

7. An adapter for a hypodermic syringe, the adapter comprising:
a thumb-contact member;
one or more spacers, each attached substantially perpendicular to the thumb-contact member; and
a plunger-contact member, attached substantially perpendicular to the spacers and facing and opposed to the thumb-contact member, the plunger-contact member incorporating a cutout adapted to being snapped onto a plunger of a hypodermic syringe.

8. The adapter of claim 7, wherein the adapter is a single molded piece.

9. The adapter of claim 8, wherein the adapter is “C”-shaped.

10. An assembly including a hypodermic syringe, the assembly comprising:
a barrel;
a plunger that comprises
a shaft comprising a proximal end and distal end,
a rubber head affixed to the distal end of the plunger and configured to form a liquid-tight seal around the inside of the barrel when the plunger is inserted into the barrel, and
a thumb rest at the proximal end of the shaft; and
an adapter affixed to the plunger, the adapter comprising
a thumb-contact member,
one or more spacers, each attached substantially perpendicular to the thumb-contact member, and
a plunger-contact member attached substantially perpendicular to the spacers and facing and opposed to the thumb-contact member, the plunger-contact member incorporating a cutout adapted to being snapped onto the plunger.
